(19) United States
(12) Patent Application Publication
(10) Pub. No.: US 2018/0226144 A1
(43) Pub. Date: Aug. 9, 2018

(54) INFORMATION PROCESSING APPARATUS, INFORMATION PROCESSING METHOD, AND NON-TRANSITORY COMPUTER READABLE STORAGE MEDIUM

(71) Applicants: YAEGAKI Bio-industry, Inc., Himeji-shi, Hyogo (JP); Godo Kaisha IP Bridge 1, Chiyoda-ku, Tokyo (JP)

(72) Inventors: Shinji Ishihara, Hyogo (JP); Masatoshi Taketani, Tokyo (JP); Kenichi Hashizume, Tokyo (JP)

(21) Appl. No.: 15/750,525

(22) PCT Filed: Aug. 12, 2016
(86) PCT No.: PCT/JP2016/073740

§ 371 Date: Feb. 6, 2018

(30) Foreign Application Priority Data
Aug. 12, 2015 (JP) 2015-159701

(51) Int. Cl. G16H 20/10; G06F 17/30; G16H 10/20

(52) U.S. Cl. CPC G16H 20/10; G16H 10/20; G06F 17/30601

(57) ABSTRACT

To enable processing to suitably recommend, among a plurality of functional materials or bacteria including one or more lactic acid bacteria that make differences in effect among individuals, appropriate ones suiting a user for each of a plurality of users.

A user information acquisitor 301 acquires user information. A recommendation target extractor 503 extracts and presents information about a functional material including one or more lactic acid bacteria to the user. A grouping unit 305 classifies a plurality of users into groups based on information about an intestinal bacterial flora. The recommendation target extractor 503 presents the information about the functional material to the user acquired by the user information acquisitor 301 based on information acquired after another user classified into the same group as the user by the grouping unit 305 takes in the functional material and an evaluation of the functional material previously done by the user.

### TECHNICAL FIELD

The present invention relates to an information processing apparatus, an information processing method, and a program.

### BACKGROUND ART

In recent years, a vast variety of supplements has been on the market. Thus, users need to select supplements to be taken in by users themselves from among such a variety of supplements.

For this purpose, there are systems that propose supplements to a user based on information input by the user or checkup results of the user (see, for example, Patent Literatures 1 and 2).

### CITATION LIST

### Patent Literature

### [Patent Literature 1]

Japanese Unexamined Patent Application Publication No. 2011-232989

### [Patent Literature 2]

Japanese Unexamined Patent Application Publication No. 2011-204194

### SUMMARY OF INVENTION

### Technical Problem

However, these conventional supplement proposing systems including those disclosed in the above Patent Literatures assume general supplements whose effect makes little difference among individuals. Thus, conventional supplement proposing systems propose supplements almost without consideration of differences in effect among individuals.

Therefore, when supplements whose effect makes much difference among individuals depending on the type of bacteria like lactic acid bacteria are to be recommended, supplements proposed by conventional supplement proposing systems may suit some users, but do not necessarily suit other users.

Such circumstances are not limited to supplements but similarly apply in a case of recommending a plurality of functional materials or bacteria including one or more lactic acid bacteria that make differences in effect among individuals.

The present invention has been made in view of such circumstances and an object thereof is to appropriately perform processing to recommend, among a plurality of functional materials or bacteria including one or more lactic acid bacteria that make differences in effect among individuals, appropriate ones suiting each of a plurality of users.

### Solution to Problem

To achieve the above object, an information processing apparatus according to an aspect of the present invention includes an acquisitor that acquires user information, a presenter that presents information about a functional material including one or more lactic acid bacteria to the user, and a classifier that classifies a plurality of users into groups based on information about an intestinal bacterial flora, wherein the presenter presents the information about the functional material to the user acquired by the acquisitor based on information acquired after another user classified into a same group as the user by the classifier takes in the functional material and an evaluation of the functional material previously done by the user.

An information processing apparatus according to another aspect of the present invention includes an acquisitor that acquires user information and a presenter that presents information about a functional material to the user, wherein the presenter presents the information about the functional material presented to the user acquired by the acquisitor based on information acquired after another user than the user takes in the functional material.

Moreover, the information acquired after the other user takes in the functional material may be an evaluation with respect to the taken functional material done after the other user takes in the functional material.

Moreover, the information acquired after the other user takes in the functional material may be an examination result of a physical examination or excrement of the other user done after the other user takes in the functional material.

Moreover, the examination result of the excrement may be an examination result of intestinal bacteria of the other user.

Moreover, the presenter may present the information about the functional material presented to the user acquired by the acquisitor based on the evaluation of the functional material previously done by the user.

Moreover, an objective acquisitor that acquires an objective of the user to take in the functional material may be included and the presenter may change the information about the functional material sent to the user in accordance with the objective acquired by the objective acquisitor.

Moreover, a classifier that classifies a plurality of users into two or more groups may be included.

The other user may be selected from the same group as the user.

Moreover, the classifier may classify users using a degree of similarity of an intestinal bacterial flora.

Moreover, the functional material may include one or more lactic acid bacteria.

An information processing method and a program according to an aspect of the present invention are a method and a program corresponding to the information processing apparatus according to an aspect of the present invention described above.

### Advantageous Effects of Invention

According to the present invention, processing to recommend, among a plurality of functional materials or bacteria including one or more lactic acid bacteria that make differences in effect among individuals, appropriate ones suiting users can be enabled for each of a plurality of users. Moreover, it becomes possible to predict the effect of functional materials or bacteria without the need for all users to take in the functional materials or bacteria so that processing to recommend functional materials or bacteria suiting each user can efficiently be performed.

### BRIEF DESCRIPTION OF DRAWINGS

FIG. 1 is a diagram showing the constitution of an information processing system.

FIG. 2 is a block diagram showing the constitution of hardware of a server of the information processing system.

FIG. 3 is a functional block diagram showing a functional constitution of the server and user terminals constituting the information processing system.

FIG. 4 is a diagram showing a concrete example of a data constitution of a question DB used by the server.

FIG. 5 is a diagram showing a concrete example of a data constitution of a material DB used by the server.

FIG. 6 is a diagram showing a concrete example of a questionnaire form displayed by the user terminal.

FIG. 7 is a diagram showing a concrete example of a data constitution of additional data for the material DB used by the server.

FIG. 8 is a flowchart illustrating first correction processing performed by the server.

FIG. 9 is a functional block diagram showing a different constitution of the functional constitutions of the server and user terminals constituting the information processing system.

FIG. 10 is a diagram showing concrete contents of characteristic factors when users are classified into a plurality of groups.

FIG. 11 is a diagram showing an overview of processing to classify users into a plurality of groups.

FIG. 12 is a diagram showing an overview of a test for one or more test members selected, from among classified users, as representatives of each group.

FIG. 13 is a diagram showing an overview of processing by the server when a new user is added.

FIG. 14 is a flowchart illustrating third correction processing performed by the server.

### DESCRIPTION OF EMBODIMENTS

Hereinafter, a first embodiment of the present invention will be described with reference to the drawings.

### First Embodiment

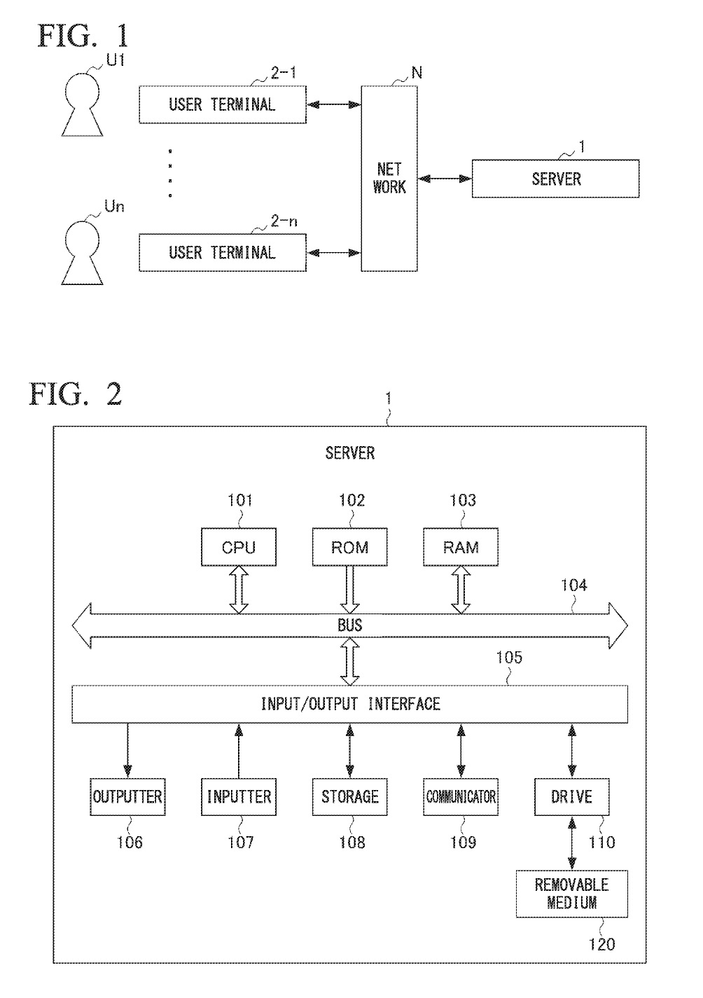

FIG. 1 is a diagram showing the constitution of an information processing system according to the first embodiment.

The information processing system according to the first embodiment has a constitution as shown in FIG. 1 so as to recommend supplements in consideration of even differences in the effect of lactic acid bacteria among individuals.

That is, the information processing system according to the first embodiment includes a server 1 as an embodiment of the information processing apparatus of the present invention and n (n is an integer of 1 or greater) user terminals 2-1 to 2-n used by n users U1 to Un, respectively. The server 1 and the user terminals 2-1 to 2-n are connected to each other via a network N such as an Internet line.

Hereinafter, when there is no need to individually distinguish the users U1 to Un and the user terminals 2-1 to 2-n, these users and user terminals are collectively called “users U” and “user terminals 2”, respectively.

The server 1 stores information to conduct a questionnaire about living body information of each of the users 2 (hereinafter, called “questionnaire form information”) and information showing supplement materials (hereinafter, called “material information”) in association with each other.

Here, “supplement materials” in this specification mean a plurality of functional materials or bacteria including one or more lactic acid bacteria that make differences in effect among individuals.

Incidentally, a functional material means a material that is not a main material of food but has a function to provide added value (nutritional intake, health maintenance and the like) to food as an indispensable material to produce food. Functional materials include probiotics materials and prebiotics materials. A probiotics material is a viable bacterium material made of a specific species and has an effect of enhancing the living body functions by multiplying useful bacteria that ameliorate the intestinal environment. Probiotics materials include, for example, viable bacteria such as lactic acid bacteria, butyric acid bacteria, and bacillus natto. A prebiotics material is an indigestible nutritive substance that guides the living body toward better health by specifically multiplying or activating useful bacteria inside the colon. Prebiotics materials include, for example, oligosaccharide, dietary fiber, and gluconic acid.

That is, bacteria that affect the intestinal bacterial flora include different types of lactic acid bacteria and bifidobacteria and these bacteria become functional materials that help specific bacteria to grow.

In response to an inquiry from the user terminal 2 desiring recommendations of supplements, the server 1 creates a questionnaire form screen based on questionnaire form information and causes the user terminal 2 to display the screen.

The user U inputs answers to the questionnaire by operating the user terminal 2 while viewing the questionnaire form screen. Information indicating answers to the questionnaire input as described above will be called “user information” below. Incidentally, the user information may contain information about the intestinal bacterial flora of the user U including the degree of similarity of the intestinal bacterial flora shown in answers to the questionnaire (hereinafter, called “intestinal bacterial flora information”).

The user terminal 2 sends the user information to the server 1 together with an identifier that uniquely identifies the user U (hereinafter, called “user identification information”).

The server 1 stores the user information in association with the user identification information.

The server 1 performs processing to recommend supplement materials suiting the user U based on user information and the relationship between questionnaire form information and material information.

Here, the recommendation means presenting, to the user U, supplement materials that suit the user U. Specifically, the recommendation means sending information to be presented to the user from the server 1 to the user terminal 2 and causing the user terminal 2 to display the information on the screen. Thus, information to be presented to the user will not be displayed on the display of the server 1.

The user U inputs an evaluation of the recommendation result (such as the effectiveness exhibited when a supplement material is actually taken in) by operating the user terminal 2. Incidentally, the method of evaluating the recommendation result is not specifically limited. For example, examination results of a physical examination carried out after taking in supplements, examination results of excrement and the like may be included. Incidentally, examination results of excrement may be determination results of the color, odor, shape and the like of the excrement.

Such information indicating an evaluation of the recommendation result from the user U is sent from the user terminal 2 to the server 1 as first feedback information.

Then, based on the first feedback information for the recommendation result from the user U, the server 1 corrects subsequent recommendation results or recommendation processing.

Such corrections are made independently of each other for each of a plurality of users U. Accordingly, in the subsequent recommendations, more appropriate supplement materials will be recommended for each of the plurality of users U. In this manner, processing to recommend, among a plurality of functional materials or bacteria including one or more lactic acid bacteria that make differences in effect among individuals, appropriate ones suiting the user U can be suitably performed for each of the plurality of users U.

However, if only first feedback information of some user U is considered for recommendation processing to that user U, the recommendation may not necessarily be appropriate for the user U. This is because the first feedback information of the user U is merely subjective information of the user U.

Thus, to make recommendations that also take objective information into consideration, the server 1 according to the first embodiment corrects subsequent recommendation results or recommendation processing based on first feedback information of other users U whose living body information is similar to that of the relevant user U, as well as first feedback information of the relevant user U.

FIG. 2 is a block diagram showing the constitution of hardware of the server 1 of the information processing system according to the first embodiment.

The server 1 includes a central processing unit (CPU) 101, a read only memory (ROM) 102, and a random access memory (RAM) 103.

The server 1 also includes a bus 104, an input/output interface 105, an outputter 106, an inputter 107, a storage 108, a communicator 109, and a drive 110.

The CPU 101 performs various kinds of processing according to a program recorded in the ROM 102 or a program loaded from the storage 108 into the RAM 103.

Data and the like needed by the CPU 101 to perform various kinds of processing are also stored in the RAM 103 when appropriate.

The CPU 101, the ROM 102, and the RAM 103 are connected to each other via the bus 104. The input/output interface 105 is also connected to the bus 104. The outputter 106, the inputter 107, the storage 108, the communicator 109, and the drive 110 are connected to the input/output interface 105.

The outputter 106 includes a display, a speaker and the like to output video and sound.

The inputter 107 includes various buttons such as a power button and operation buttons to input various kinds of information according to user's instruction operations.

The storage 108 includes a hard disk, a dynamic random access memory (DRAM) or the like to store data of various kinds of information such as material information and user information.

The communicator 109 controls communication with the user terminal 2 via the network N including the Internet.

A removable medium 120 made of a magnetic disk, an optical disk, a magneto-optical disk, or a semiconductor memory is appropriately inserted into the drive 110. A program read by the drive 110 from the removable medium 120 is installed in the storage 108 when necessary. The removable medium 120 can also store various kinds of data such as material information and user information stored in the storage 108 in a similar manner to the storage 108.

Though not shown, each of a plurality of user terminals 2 has a constitution of hardware similar to that of the server 1 in FIG. 2.

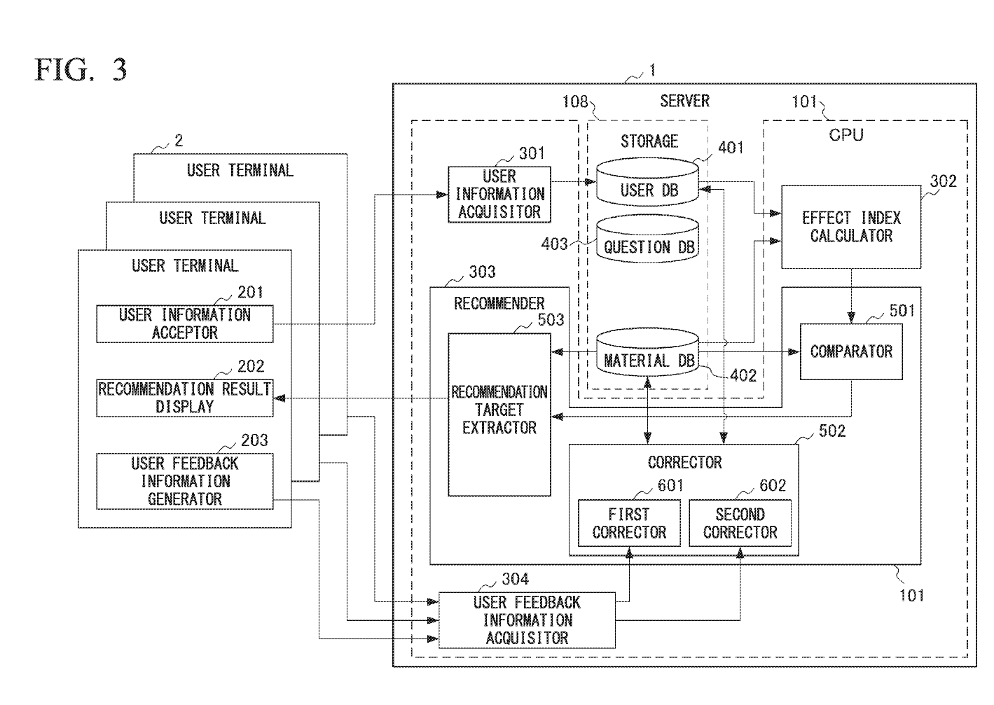

FIG. 3 is a functional block diagram showing a functional constitution of the server 1 and the user terminals 2 in FIG. 1.

In the CPU 101 of the server 1, a user information acquisitor 301, an effect index calculator 302, a recommender 303, and a user feedback information acquisitor 304 function.

The recommender 301 includes a comparator 501, a corrector 502, and a recommendation target extractor 503.

The corrector 502 includes a first corrector 601 and a second corrector 602.

Moreover, in a portion of the storage 108, a user database (hereinafter, abbreviated as a “user DB”) 401, a material database (hereinafter, abbreviated as a “material DB”) 402, and a question database (hereinafter, abbreviated as a “question DB”) 403 are stored.

In the user terminal 2, a user information acceptor 201, a recommendation result display 202, and a user feedback information generator 203 function.

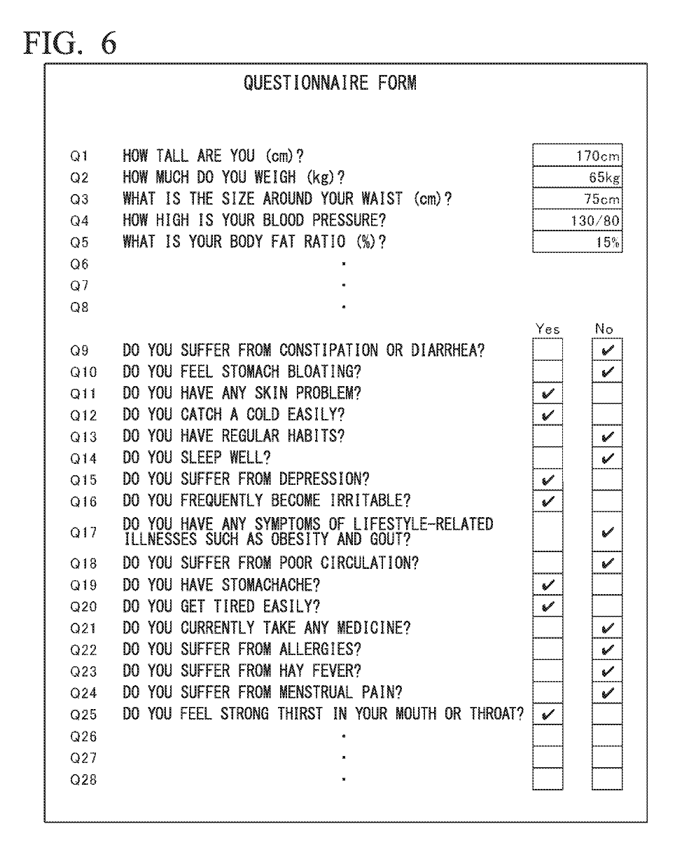

To allow answers of the user U to a questionnaire to be input as user information, the user terminal 2 displays, for example, a screen as shown in FIG. 6.

FIG. 6 is an example of the questionnaire form screen to input user information.

The user U inputs his/her answers to the questionnaire by operating the user terminal 2 while following the questionnaire form screen in FIG. 6.

The user information acquisitor 301 accepts the answers of the user U to the questionnaire as user information.

The user information is sent from the user terminal 2 to the server 1 together with the identification information of the user U.

The user information acquisitor 301 of the server 1 acquires the user information from the user terminal 2. Then, the user information acquisitor 301 exercises control to cause the user DB 401 to store the user information in association with user identification information.

In this manner, corresponding user information is stored for each piece of user identification information in the user DB 401.

The effect index calculator 302 calculates the index of effect (hereinafter, called a “score”) for the user U for each supplement material based on the user information stored in the user DB 401 and supplement material information stored in the material DB 402.

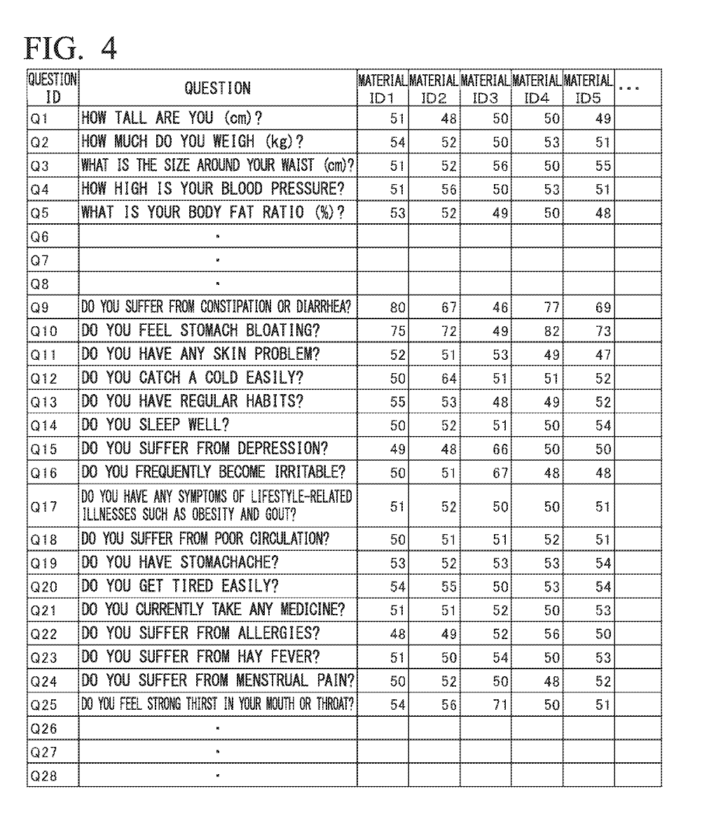

FIG. 4 is an example of, among pieces of information stored in the material DB 402, information indicating the relevance between a supplement material and each question in a questionnaire.

The information in FIG. 4 contains a relevance degree between each question displayed on the questionnaire form screen in FIG. 6 and each supplement material. The relevance increases with an increasing relevance degree.

Each question displayed on the questionnaire form screen in FIG. 6 is identified by the question ID and the question item. Each supplement material is identified by the material ID.

As shown in FIG. 4, for example, the relevance degree between the question (hereinafter, called “question Q1”) identified by the question ID “Q1” and the question item “How tall are you (cm)?” and the material of the material ID 1 is set to “51”. Meanwhile, the relevance degree between the question Q1 and the material of the material ID 2 is set to “48”. Thus, it is shown that the material of the material ID 1 has a higher relevance degree with the question Q1, that is, the height than the material of the material ID 2. That is, it is shown that the material of the material ID 1 has a larger difference in effectiveness at different heights than the material of the material ID 2.

Moreover, the relevance degree between the question (hereinafter, called “question Q9”) identified by the question ID “Q9” and the question item “Do you suffer from constipation or diarrhea?” and the supplement material identified by the material ID 1 is set to “80”. Meanwhile, the relevance degree between the question Q9 and the supplement material identified by the material ID 2 is set to “67”. Thus, it is shown that the material of the material ID 1 has a higher relevance degree with the question Q9, that is, constipation or diarrhea than the material of the material ID 2. That is, it is shown that the material of the material ID 1 has a greater effect of improving constipation and the like than the material of the material ID 2.

Returning to FIG. 3, the effect index calculator 302 calculates, as a score, a value obtained by adding up a value based on the relevance degree between each question and each material for all questions for each supplement material.

Here, “value based on the relevance degree between each question and each material” will be described

The questions shown in FIG. 4 are roughly divided into those, like the question Q1, that require the user to input a numerical value (hereinafter, called “numerical value input questions”) and those, like the question Q9, that are answered by Yes/No (hereinafter, called “multiple-choice questions”).

In a numerical value input question, the value determined by a predetermined operation using an input numerical value and the relevance degree between each question and each material becomes a “value based on the relevance degree between each question and each material”.

For example, a difference between the numerical value input by a user and the average numerical value of all users is determined and then, a value obtained by multiplying the difference by the relevance degree between the relevant question and each material may be set as the “value based on the relevance degree between each question and each material”.

Specifically, it is assumed that, for example, “170” is input as the answer to the question Q1. In this case, if the average of all users regarding the question Q1 is, for example, “165”, the difference from the average is “+5”. The relevance degree between the question Q1 and the material ID 1 is “51” and thus, the value based on the relevance degree between the question Q1 and the material ID 1 becomes “251 (=+5×51)”. Meanwhile, the relevance degree between the question Q1 and the material ID 2 is “48” and thus, the value based on the relevance degree between the question Q1 and the material ID 2 becomes “240 (=+5×48)”.

In a multiple-choice question, by contrast, if the answer is Yes, the “relevance degree itself between each question and each material” becomes the “value based on the relevance degree between each question and each material”. If the answer is No, the value obtained by subtracting the “relevance degree between each question and each material” from 100 becomes the “value based on the relevance degree between each question and each material”.

For example, if the answer is Yes in the question Q9, “80” becomes the “value based on the relevance degree between each question and each material”. If the answer is No, “20 (=100−80)” becomes the “value based on the relevance degree between each question and each material”.

Thus, in the first embodiment, the “value based on the relevance degree between each question and each material” is calculated for each question regarding one predetermined material and the total value of the “value based on the relevance degree between each question and each material” for each question is calculated as a score of the relevant material.

However, the score calculation technique is not limited to the example of the first embodiment as a matter of course and any technique may be used.

Returning to FIG. 3, the comparator 501 compares the calculated score with a preset threshold for each supplement material with reference to the material DB 402.

The recommendation target extractor 503 extracts a supplement material having a score exceeding the threshold as a recommendation target with reference to the material DB 402.

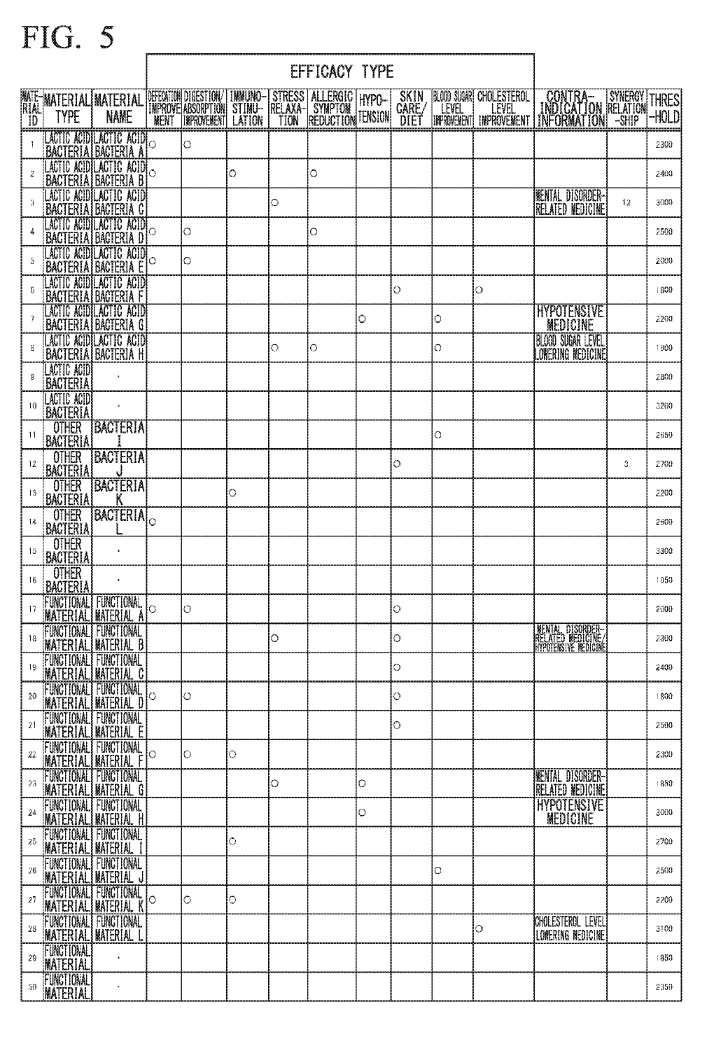

Among pieces of information stored in the material DB 402, information referred to by the comparator 501 and the recommendation target extractor 503 is, for example, information shown in FIG. 5.

FIG. 5 is an example of information of supplement materials stored in the material DB 402.

In FIG. 5, a predetermined row corresponds to one predetermined supplement material. In the relevant row, the material ID, material type, material name, effect, contraindication information, synergy relationship, and threshold of the corresponding supplement material are each stored.

Specifically, for example, in the example of FIG. 5, the threshold of lactic acid bacteria A of the material ID 1 is set to 2300 and the threshold of lactic acid bacteria B of the material ID 2 is set to 2400.

Therefore, for example, if the score of the material ID 1 is 2200 and the score of the material ID 2 is 2500, the lactic acid bacteria A of the material ID 1 have a score less than the threshold and are thus excluded from recommendations, but the lactic acid bacteria B of the material ID 2 have a score exceeding the threshold and are thus to be recommended.

In the example of FIG. 5, it is clear that lactic acid bacteria C of the material ID 3 and lactic acid bacteria J of the material ID 12 have a synergy relationship. In such a case, for example, the average value of the scores of both may be multiplied by a predetermined coefficient such as 1.5 before being compared with the average value of the thresholds of both. Here, the predetermined coefficient may be a different value for each synergy relationship.

Even if a supplement material has a score exceeding the threshold, the recommendation target extractor 503 excludes, from recommendations, a supplement material that should be contraindicated in connection with the user.

Information as to whether to contraindicate a material is contraindication information. In the example of FIG. 5, that mental disorder-related medicine should be contraindicated for lactic acid bacteria C of the material ID 3 is stored as contraindication information.

Here, if the answer of Q21 in the example of FIG. 6 is Yes, the user information acceptor 201 of the user terminal 2 may allow the user to input by having a drug name field displayed. If a contraindicated drug is input into the drug name field or the drug name is unknown and the possibility of contraindication cannot be excluded, the recommendation target extractor 503 excludes a supplement material from recommendations even if the supplement material has a score exceeding the threshold.

An objective acquisitor 504 acquires an objective Trg of taking in a functional material (hereinafter, simply called an “objective”).

The recommendation result display 202 of the user terminal 2 displays one or more supplement materials extracted by the server 1 as recommendation targets to the user to prompt the user to purchase the supplement materials.

The server 1 acquires information of the purchase or non-purchase from the user terminal 2, and uses the information for remind advertisements or feedback. The user terminal 2 may also hold the relevant information.

When a predetermined period passes after the user U purchases a supplement, the user feedback information generator 304 causes the user U to input an evaluation of effectiveness about one type of efficacy or more to generate first feedback information based on the input contents.

The first feedback information contains accumulated information of past evaluations by the user U.

To obtain cooperation from the user U to generate first feedback information, the user feedback information generator 304 may provide, for example, remuneration such as a cash refund.

The user feedback information generator 304 may also display a remind message as appropriate while the user U does not input.

The user feedback information acquisitor 304 of the server 1 acquires first feedback information generated by each of the user terminals 2.

Based on first feedback information to a recommendation result from at least a part of the plurality of user terminals 2, the corrector 502 corrects subsequent processing of the recommender 303 for a predetermined user terminal 2. Here, “subsequent” means the time when first feedback information is acquired or thereafter. That is, the corrector 502 corrects processing performed at time T1 or thereafter based on first feedback information acquired before time T1.

In the first embodiment, the corrector 502 corrects the relevance degree for each question ID or the threshold for materials contained in the first feedback information as corrections of processing of the recommender 303 for the predetermined user terminal 2.

Specifically, the first corrector 601 of the corrector 502 corrects the relevance degree for each question ID of the predetermined user terminal 2 about materials fed back by the predetermined user terminal 2 based on first feedback information sent from the predetermined user terminal 2 and corresponding user information.

Based on first feedback information sent from each of the plurality of user terminals 2, the second corrector 602 corrects subsequent recommendation results or processing of the recommender 303 by correcting the threshold of a predetermined material of the predetermined user terminal 2.

Next, a sequence of processing (hereinafter, called “first correction processing”) from the acquisition of user information about the user U to corrections of recommendation results or recommendation processing by server 1 based on first feedback information will be described.

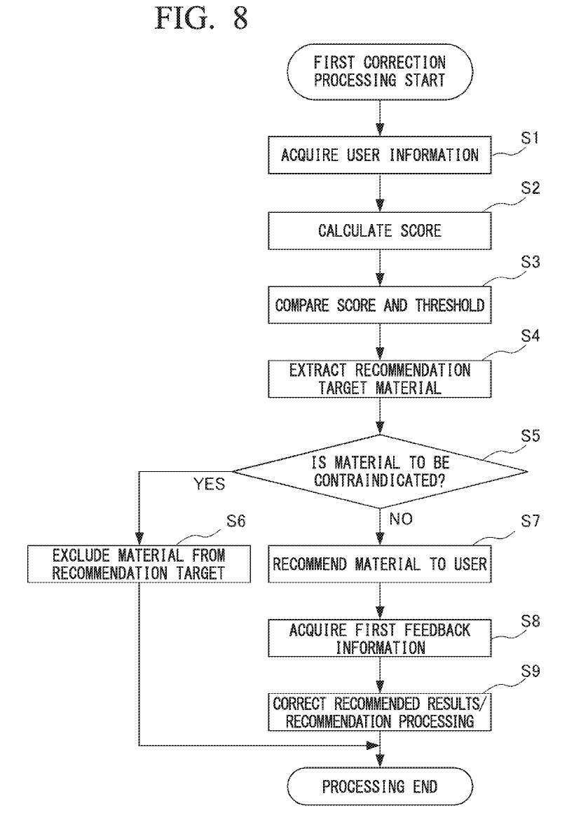

FIG. 8 is a flowchart illustrating personal correction processing performed by the server 1 in FIG. 1.

In step S1, the user information acquisitor 301 acquires user information accepted by the user information acceptor 201 of the user terminal 2 and causes the user DB 401 to store the user information.

In step S2, the effect index calculator 302 calculates the score of a supplement material for the user U for each supplement material based on the user information stored in the user DB 401 and material information stored in the material DB 402 in advance.

In step S3, the comparator 501 compares the score calculated by the effect index calculator 302 with the preset threshold for each supplement material with reference to the material DB 402.

In step S4, the recommendation target extractor 503 extracts a supplement material having a score exceeding the threshold as a recommendation target with reference to the material DB 402.

In step S5, the recommendation target extractor 503 determines whether any supplement material whose score exceeds the threshold is to be contraindicated in connection with the user U.

If it is determined that the supplement material is to be contraindicated in connection with the user U in step S5, a determination of YES is made in step S5 and the processing proceeds to step S6. If it is determined that the supplement material is not to be contraindicated in connection with the user U, on the other hand, a determination of NO is made in step S5 and the processing proceeds to step S7.

In step S6, the supplement material determined to be contraindicated in connection with the user U is excluded from recommendations to the user U.

In step S7, the recommendation target extractor 503 performs processing to recommend the supplement material to the user U. At this point, the recommendation result display 202 of the user terminal 2 displays a message of supplement material recommendation to the user U by causing the user terminal 2 to display the message.

In step S8, when the recommended supplement material is evaluated by the user U, the user feedback information acquisitor 304 acquires the evaluation by the user U as first feedback information.

In step S9, the first corrector 601 corrects subsequent recommendation results or recommendation processing to the user U based on the first feedback information. This terminates the first correction processing.

### Second Embodiment

The constitution of an information processing system according to a second embodiment and the hardware constitution of a server of the information processing system are similar to those in the first embodiment.

The server 1 in FIG. 1 classifies the user U into one or more groups among a plurality of groups based on at least information containing intestinal bacterial flora information of the acquired user information. Here, characteristic factors (hereinafter, abbreviated as “grouping identification factors”) for classification are not specifically limited. In the second embodiment, users are classified as described below. That is, information obtained from answers to questions to each user U is used as grouping identification factors.

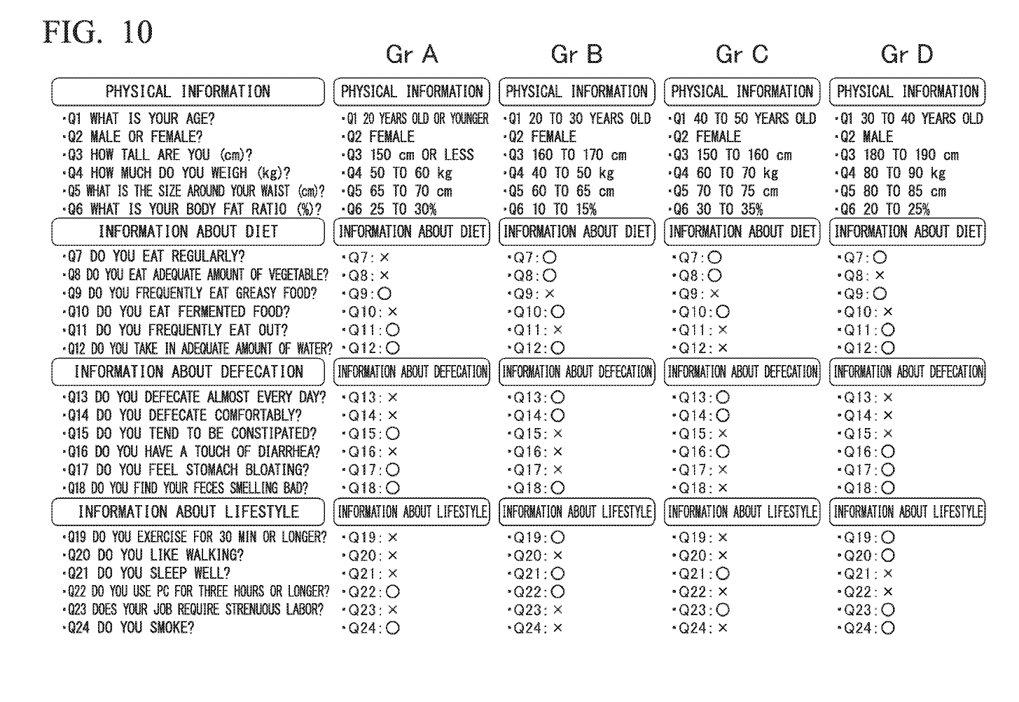

Specifically, information obtained from answers to questions to each user U is divided into four categories by nature to classify users U with similar answer contents into the same grouping. In the second embodiment, information obtained from answers to questions to each user U is divided by nature into four categories of “physical information”, “information about diet”, “information about defecation”, and “information about lifestyle”. Concrete contents of the grouping characteristic factors will be described below with reference to FIG. 10.

The server 1 selects a test member from among one or more users U belonging to each group of a plurality of groups. The test member of a predetermined group represents the relevant group and takes in the supplement material recommended by the server 1 based on user information of the test member. In this manner, the effect (whether any change for the better occurs) of the relevant supplement material on the group is tested.

When performing processing to recommend the supplement material suiting the relevant test member, the server 1 may, for example, extract the objective of the relevant test member to take in the supplement material as a recommendation target. Specifically, by setting the objective Trg of, for example, lowering the body fat ratio, the server 1 can extract a supplement material having an effect of achieving the objective Trg as a recommendation target.

Here, users U belonging to the same group are likely to have constitutional commonality such as similar patterns of the intestinal bacterial flora. Thus, the effect of a supplement material on the test member representing a group is likely to be similar to the effect of the relevant supplement material on other users U classified into the group to which the test member belongs.

Accordingly, if the test member having taken in the supplement material recommended by the server 1 experiences a change for the better due to intake of the supplement material, the server 1 makes a correction to increase the probability of extracting and recommending the supplement material to all the users U classified into the group to which the test member belongs.

On the other hand, if the test member experiences no change for the better due to intake of the supplement material, the server 1 makes a correction to decrease the probability of extracting and recommending the supplement material to all the users U classified into the group to which the test member belongs.

Accordingly, it becomes possible to predict the effect of a supplement material without the need for all users U to take in the supplement material so that the server 1 can efficiently perform processing to recommend a supplement material suiting each user U.

Incidentally, the determination method for determining whether any change for the better has occurred to the test member is not specifically limited. For example, a determination method including the following first to third steps may be used. The first step is a step of acquiring, by the user terminal 2, information indicating an evaluation (such as the effectiveness exhibited when a supplement material is actually taken in) of recommendation results by the server 1 input by the relevant test member. The second step is a step of sending, by the user terminal 2, the acquired relevant information to the server 1 as second feedback information. The third step is a step of determining by the server 1 whether any change for the better has occurred to the relevant test member based on the second feedback information.

It is also possible to adopt a determination method by which the relevant test member actually has an examination in a predetermined examination organization, the server 1 acquires the result of the examination as second feedback information, and whether any change for the better has occurred to the test member is determined based on the second feedback information.

Thus, the second feedback information can be used for corrections that vary the probability of the server 1 extracting and recommending a supplement material to all users U of the group to which the relevant test member belongs.

Further, the second feedback information can be used to correct user information of any user U who is not selected as a test member. For example, the server 1 corrects user information of the user U based on second feedback information. The server 1 performs recommendation processing of supplement materials based on corrected user information about the user U. In this case, the server 1 can further correct user information about the user U based on first feedback information from the user U.

Here, the content of the first feedback information to correct user information is not specifically limited, but in the second embodiment, results of a questionnaire to the user U are used.

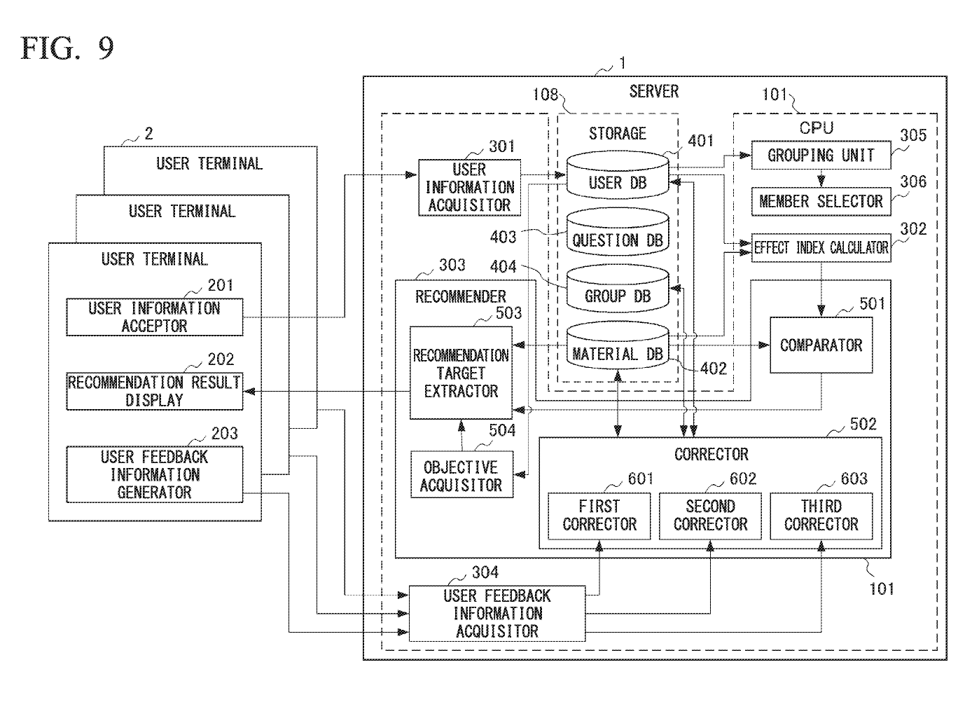

FIG. 9 is a functional block diagram showing a different constitution of the functional constitutions of the server and user terminals constituting the information processing system.

In the CPU 101 of the server 1, like in the first embodiment in FIG. 3, the user information acquisitor 301, the effect index calculator 302, the recommender 303, and the user feedback information acquisitor 304 function. Further, in the second embodiment, a grouping unit 305 and a member selector 306 function.

The recommender 301 includes, like in the first embodiment in FIG. 3, the comparator 501, the corrector 502, the recommendation target extractor 503, and the objective acquisitor 504.

The corrector 502 includes, like in the first embodiment in FIG. 3, the first corrector 601 and the second corrector 602. In the second embodiment, a third corrector 603 is further included in the corrector 502.

Moreover, in a portion of the storage 108, like in the functional constitution of FIG. 3, the user DB 401, the material DB 402, and the question DB 403 are stored. Further, in the functional constitution of FIG. 9, a group DB 404 is additionally stored in a portion of the storage 108.

In the user terminal 2, like in the functional constitution of FIG. 3, the user information acceptor 201, the recommendation result display 202, and the user feedback information generator 203 function.

The grouping unit 305 classifies each of a plurality of users U into one or more groups among a plurality of groups based on user information of each of the plurality of users U. As described above, the content of the grouping identification factors acting as characteristic factors when classifying the user U into one or more groups among the plurality of groups is not specifically limited. In the second embodiment, information obtained from answers to question items to each user U illustrated in FIG. 4 is used as grouping identification factors for classification. Moreover, the method for processing groupings is not specifically limited. For example, groupings can be done by an algorithm based on past data or machine learning.

Incidentally, information about classification of users U by the grouping unit 305 (hereinafter, called “grouping information”) including grouping identification factors is stored in the group DB 404.

Here, a concrete example of the grouping identification factors in the second embodiment will be described.

FIG. 10 shows an example of the grouping identification factors in the second embodiment.

Specifically, the grouping unit 305 classifies information obtained from answers to questions to each user U into four types of information, “physical information”, “information about diet”, “information about defecation”, and “information about lifestyle” and information obtained from answers to questions to each user U is used as grouping identification factors for classification.

For example, a question item (question Q1) of “What is your age?” can be set regarding “physical information” to be a grouping identification factor. Then, four choices of “19 years old or younger”, “20 to 29 years old”, “30 to 39 years old”, and “40 years old or older” are set in advance as the answer to the question item. Then, the user U corresponding to “19 years old or younger” can be classified into a group A, the user U corresponding to “20 to 29 years old” into a group B, the user U corresponding to “30 to 39 years old” into a group C, and the user U corresponding to “40 years old or older” into a group D.

Moreover, for example, a question item (question Q7) of “Do you eat regularly?” can be set regarding “information about diet” to be a grouping identification factor. Then, two choices of “Yes” and “No” are set in advance as the answer to the question item. Accordingly, the user U corresponding to, for example, “Yes” can be classified into one of the groups B to D and the user U corresponding to “No” can be classified into the group A.

In addition, regarding “information about defecation”, and “information about lifestyle” acting as grouping identification factors in the present embodiment, each of the question items 13 to 24 can similarly be set.

However, the number and content of grouping characteristic factors are not limited to the above examples. Information other than the above four types of information can be selected as identification factors.

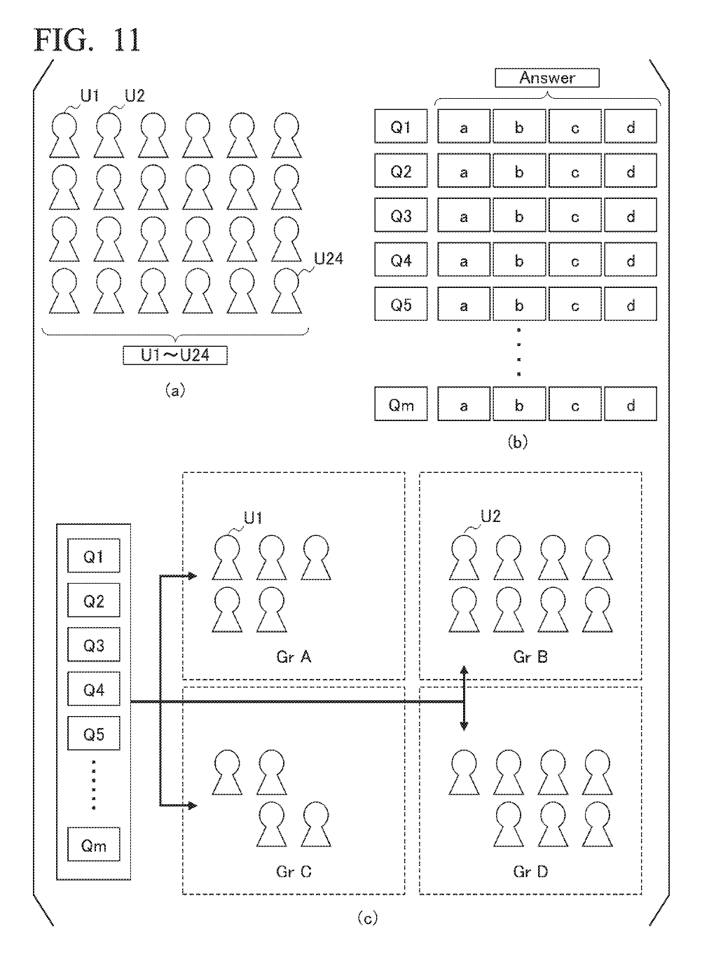

FIG. 11 shows an overview of processing in which the grouping unit 305 classifies users U. FIG. 11(a) shows a case where 24 users U (users U1 to U24) are present. In this case, the grouping unit 305 classifies each of the users U into one of the groups A to D using, as grouping identification factors, information obtained from answers of the user U to question items (question items Q1 to Qm (m is an integer of 1 or greater)) shown in FIG. 11(b). Specifically, for example, as shown in FIG. 11(c), the user U1 is classified into the group A and the user U2 is classified into the group B.

In some cases, however, one user U may be classified into a plurality of groups or a user U may move between groups (reclassification).

In this manner, each of the users U1 to U24 is classified into one of the groups A to D by the grouping unit 305.

When, as described above, each of the plurality of users U is classified into one or more groups among the plurality of groups, the server 1 selects a test member from among one or more users U belonging to each of the plurality of groups.

Returning to FIG. 9, the member selector 306 of the server 1 selects a test member from among one or more users U classified into the plurality of groups by the grouping unit 305 and belonging to a group, for each of the plurality of groups.

The test member selected by the member selector 306 represents the group to which the relevant test member belongs and takes in the supplement material extracted and recommended by the recommendation target extractor 503 based on user information of the relevant test member. In this manner, the effect (whether any change for the better occurs) of the relevant supplement material on the group is tested.

When recommending a supplement material suiting the relevant test member, the recommendation target extractor 503 of the recommender 303 can recommend the supplement material corresponding to the objective Trg with which the relevant test member takes in the supplement material. Here, the content of the objective Trg is not specifically limited. If, for example, the objective Trg is to lower the body fat ratio, the recommendation target extractor 503 can extract and recommend a supplement material having an effect of lowering the body fat ratio, which is the objective acquired by the objective acquisitor 504.

The supplement material recommended by the recommendation target extractor 503 will be taken in by the test member. If the test member experiences a change for the better due to intake of the relevant supplement material, the third corrector 603 makes a correction to increase the probability of the relevant supplement material being extracted and recommended by the recommendation target extractor 503 to users U classified into the group to which the test member belongs.

On the other hand, the test member may not experience a change for the better even if the supplement material recommended by the recommendation target extractor 503 is taken in. In this case, the third corrector 603 makes a correction to decrease the probability of the relevant supplement material being extracted and recommended by the recommendation target extractor 503 to all users U classified into the group to which the relevant test member belongs.

Incidentally, information about the supplement material extracted by the recommendation target extractor 503 is sent to the user terminal 2 via a transmitter (not shown) of the server 1 as information allowed to be displayed on the screen of the user terminal 2.

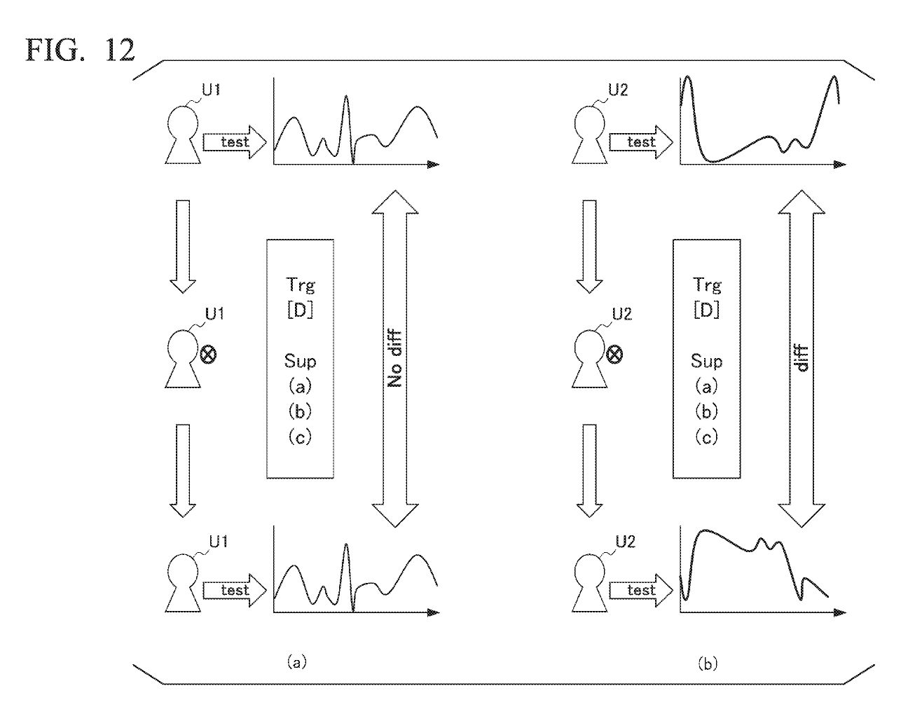

FIG. 12 shows an overview of a test on the test members.

If the objective Trg of the user U1 as a test member is an objective TrgD, the recommendation target extractor 503 extracts supplement materials a to c suiting the user U1 as recommendation targets to achieve the objective TrgD.

Then, the user U1 takes in the supplement materials a to c extracted and recommended by the recommendation target extractor 503. Accordingly, the effect (whether any change for the better occurs) caused by intake of the relevant supplement materials a to c is tested.

FIG. 12(a) shows an example in which no change for the better occurs.

In this case, the third corrector 603 determines that the intake of the supplement materials a to c by the user U1 does not have the effect of achieving the objective TrgD of the user U1. Accordingly, the third corrector 603 makes a correction to decrease the probability of the relevant supplement materials a to c being extracted and recommended by the recommendation target extractor 503 to all users U classified into the group A to which the user U1 belongs.

If the objective Trg of the user U2 as a test member is similarly the objective TrgD like the user U1, the recommendation target extractor 503 extracts, like the user U1, the supplement materials a to c suiting the user U2 as recommendation targets to achieve the objective TrgD.

Then, the user U2 takes in the supplement materials a to c extracted and recommended by the recommendation target extractor 503. Accordingly, the effect (whether any change for the better occurs) caused by intake of the relevant supplement materials a to c is tested.

FIG. 12(b) shows an example in which a change for the better occurs.

In this case, the third corrector 603 makes a correction to increase the probability of the relevant supplement materials a to c being extracted and recommended by the recommendation target extractor 503 to all users U classified into the group B to which the user U2 belongs.

In this manner, based on results of the test on test members, the probability of supplement materials being extracted and recommended by the recommendation target extractor 503 is varied. Accordingly, the possibility of recommending to the user U is increased for supplement materials whose effect is expected to be large and, on the other hand, the possibility of recommending to the user U is decreased for supplement materials whose effect is expected to be small.

FIG. 11 shows an example in which the total number of users U is 24, but a new user U may newly be added.

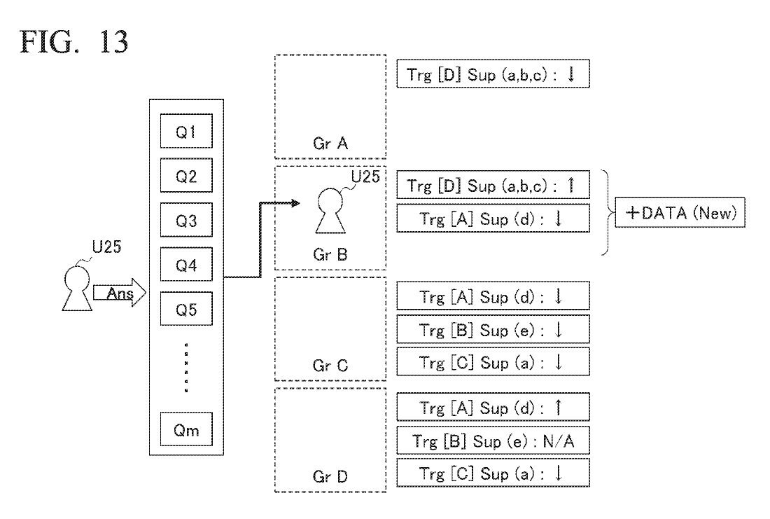

FIG. 13 is a diagram showing an overview of processing by the server 1 when a new user U25 is added.

As shown in FIG. 13, the new user U25 answers the question items Q1 to Qm written in the questionnaire by operating a user terminal 2-25. The answers by the new user U25 are acquired by the user information acquisitor 301 and stored in the user DB 401 as user information about the new user U25.

Based on information acquired from the answers of the new user U25, the grouping unit 305 of the server 1 classifies the new user U25 into one or more groups A to D. In the example of FIG. 13, the new user U25 is classified into the group B.

At this point, the first corrector 601 of the server 1 corrects user information about the new user U25 stored in the user DB 401 based on grouping information stored in the group DB 404 and material information stored in the material DB 402.

Specifically, for example, as shown in FIG. 13, the probability of the supplement materials a to c being extracted and recommended by the recommendation target extractor 503 may be set high in the group B to achieve the objective TrgD. In this case, if the objective Trg of the new user U25 is the objective TrgD, the first corrector 602 corrects user information about the new user U25 such that the possibility of the supplement materials a to c being recommended to the new user U25 increases.

Moreover, the probability of a supplement material d being extracted and recommended by the recommendation target extractor 503 may be set low in the group B to achieve an objective TrgA. In this case, if the objective Trg of the new user U25 is the objective TrgA, the first corrector 602 corrects user information about the new user U25 such that the possibility of the supplement material d being recommended to the new user U25 decreases.

Next, a sequence of processing (hereinafter, called “second correction processing”) by the server 1 from the classification of users U into one or more groups to corrections of the probability of a material being extracted and recommended based on second feedback information will be described.

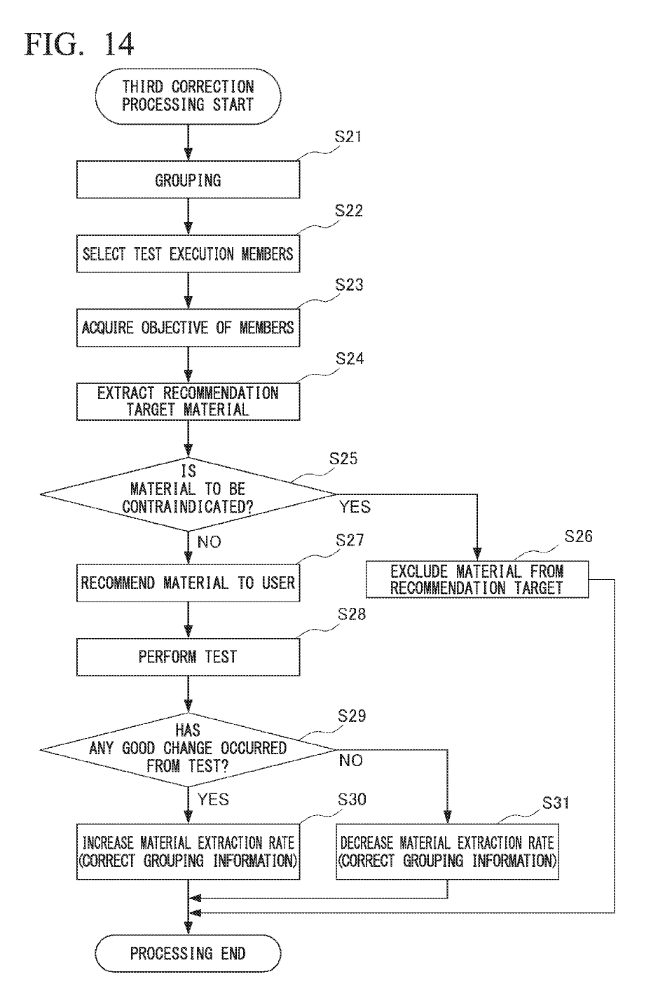

FIG. 14 is a flowchart illustrating third correction processing performed by the server 1 in FIG. 1.

In step S21, the grouping unit 305 classifies users U into a plurality of groups based on user information about the users U.

In step S22, the member selector 306 selects one or more users U as test members for each of the plurality of groups from among the users U classified into the plurality of groups by the grouping unit 305.

In step S23, the objective acquisitor 504 acquires the objective Trg of the relevant test member.

In step S24, the recommendation target extractor 503 extracts a supplement material having an effect of achieving the objective Trg of the relevant test member as a recommendation target.

In step S25, it is determined whether the supplement material extracted as a recommendation target is to be contraindicated in connection with the user. Accordingly, if it is determined that the supplement material is to be contraindicated in connection with the user, a determination of YES is made in step S25 and the processing proceeds to step S26.

In step S26, the supplement material determined to be contraindicated in connection with the user U is excluded from recommendations to the user U.

If, in step S25, a supplement material extracted to be recommended is determined not to be contraindicated in connection with the user, a determination of NO is made in step S25 and the processing proceeds to step S27.

In step S27, the recommendation target extractor 503 performs processing to recommend the supplement material to the user U. At this point, the recommendation result display 202 of the user terminal 2 displays a message of supplement material recommendation to the user U by causing the user terminal 2 to display the message.

In step S28, the test member selected by the member selector 306 represents the group to which the relevant test member belongs and takes in the supplement material recommended. Accordingly, the effect of the relevant supplement material is tested.

In step S29, the third corrector 603 determines whether the test member who has taken in the supplement material extracted by the recommendation target extractor 503 experiences a change for the better due to the intake of the supplement material. If it is determined that the test member experiences a change for the better, a determination of YES is made in step S29 and the processing proceeds to step S30.

In step 30, the third corrector 603 makes a correction to increase the probability of the relevant supplement material being extracted and recommended by the recommendation target extractor 503 to all users U classified into the group to which the relevant test member belongs. This terminates the third correction processing.

If, in step S29, it is determined by the third corrector 603 that the test member experiences no change for the better, a determination of NO is made in step S29 and the processing proceeds to step S31.

In step S31, the third corrector 603 makes a correction to decrease the probability of the relevant supplement material being extracted and recommended by the recommendation target extractor 503 to all users U of the group to which the relevant test member belongs. This terminates the third correction processing.

Incidentally, the present invention is not limited to the above embodiments, and modifications and improvements within the scope capable of achieving the object of the present invention are included in the present invention.

For example, correction targets of a corrector are not limited to those in the above embodiments and, for example, the algorithm of recommendation may be corrected as a correction of processing of the recommender of a predetermined user terminal. Moreover, for example, without correcting the processing itself of the recommender of a predetermined user terminal, a recommendation result of the predetermined user terminal may be corrected after being output.

Based on feedback information to a recommendation result from at least a portion of a plurality of users, the corrector can correct subsequent recommendation results or processing of the recommender for predetermined users.

Moreover, information of FIGS. 4 to 7 is only intended for illustration.

In addition to the question items illustrated in FIG. 4, for example, question items like “What is your age?” and “Male or female?” can be included as question items in FIG. 4 regarding physical information. Moreover, question items like “Do you eat regularly?”, “Do you eat adequate amount of vegetable?”, “Do you frequently eat greasy food?”, “Do you eat fermented food?”, “Do you frequently eat out?” and “Do you take in adequate amount of water?” can be included as question items regarding information about diet. Moreover, question items like “Do you defecate almost every day?”, “Do you defecate comfortably?” and “Do you find your feces smelling bad?” can be included as question items regarding information about defecation. Moreover, question items like “Do you exercise for 30 min or longer?”, “Do you like walking?”, “Do you use PC for three hours or longer?”, “Does your job require strenuous labor?” and “Do you smoke?” can be included as question items regarding information about lifestyle.

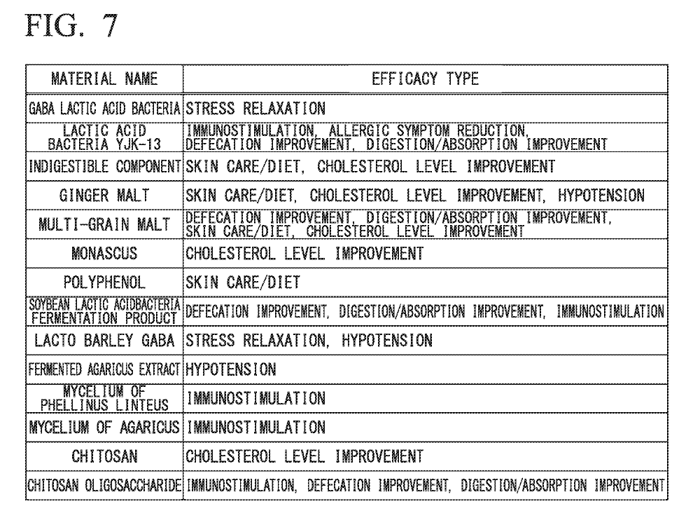

FIG. 7 is a concrete example of data for adding information of supplement materials for the material DB 402.

The material name of the data may be inherited as the material name of information of supplement materials after the supplement materials are added. The type of efficacy of the data may be adapted like dividing the data into column information for each type of efficacy of information of supplement materials after the supplement materials are added.

Specifically, for example, in the material DB 402, information such as “stress relaxation” may be added as additional information to the type of efficacy of the material name “GABA lactic acid bacteria”. Moreover, information such as “immunostimulation, allergic symptom reduction, defecation improvement, and digestion/absorption improvement” may be added as additional information to the type of efficacy of the material name “lactic acid bacteria YJK-13”. In addition, information about the type of efficacy corresponding to each material name illustrated in FIG. 7 can be added.

When information about a supplement material is added, an additional material ID column may be added for information of relevance between the supplement material and each question in a questionnaire so that processing to maintain consistency may be performed by setting appropriate relevance degrees as initial values.

Moreover in the above embodiments, the information processing apparatus to which the present invention is applied has been described as a server, but is not specifically limited to the server as long as the information processing apparatus can perform the sequence of processing described above.

Moreover, the sequence of processing described above may be performed by hardware or software.

In other words, the functional constitutions in FIGS. 3 and 9 are only an illustration and not specifically limited. That is, the information processing apparatus only needs to have a function capable of performing the sequence of processing described above as a whole, and which functional block to use to implement the above function is not limited to the examples of FIGS. 3 and 9.

One functional block may be constituted as hardware alone, software alone, or a combination of hardware and software.

The location of a functional block is not limited to the above examples of FIGS. 3 and 9 and at least a portion of the server functions may be transferred to the user terminal or another apparatus (not shown) or conversely, at least a portion of the user terminal functions may be transferred to the server or another apparatus (not shown).

When a sequence of processing is performed by software, a program constituting the software is installed on a computer and the like from a network or a recording medium.

The computer may be a computer embedded in dedicated hardware. Moreover, the computer may be a computer, for example, a general-purpose personal computer capable of executing various functions by installing various programs.

The recording media including such a program include not only the removable medium 120 in FIG. 2 distributed separately from the apparatus body to provide a program to the user, but also recording media and the like provided to the user U while being embedded in the apparatus body. The removable medium 120 includes, for example, a magnetic disk (including a floppy disk), an optical disk, or a magneto-optical disk. The optical disk includes, for example, a compact disk-read only memory (CD-ROM) or a digital versatile disk (DVD). The magneto-optical disk includes a mini-disk (MD) or the like. Recording media provided to the user by being embedded in the apparatus body include, for example, the ROM 102 in FIG. 2 in which programs are recorded, and a hard disk included in the storage 108 in FIG. 2.

In the present specification, steps describing programs recorded in a recording medium include not only processing performed chronologically in the order thereof, but also processing performed not necessarily chronologically but performed in parallel or individually.

Moreover, in this specification, terms of the system mean an overall apparatus including a plurality of apparatuses or a plurality of units.

To sum up, an information processing apparatus to which the present invention is applied only needs to have the constitution described below and various embodiments including the above embodiments can be implemented.

That is, an information processing apparatus to which the present invention is applied includes:

an acquisitor (for example, the user information acquisitor 301 in FIG. 9) that acquires user information;

a presenter (for example, the recommendation target extractor 503 in FIG. 9) that presents information about a functional material including one or more lactic acid bacteria to the user; and

a classifier (for example, the grouping unit 305 in FIG. 9) that classifies a plurality of users into groups based on information about an intestinal bacterial flora,

wherein the presenter presents, to the user acquired by the acquisitor, information about the functional material based on:

information (for example, second feedback information) acquired after other users classified into the same group as the user by the classifier take in the functional material; and

an evaluation (for example, first feedback information) of the functional material previously done by the user.

Accordingly, processing to recommend, among a plurality of functional materials or bacteria including one or more lactic acid bacteria that make differences in effect among individuals, appropriate ones suiting the user can be suitably enabled for each of a plurality of users. Moreover, it becomes possible to predict the effect of functional materials or bacteria without the need for all users to take in the functional materials or bacteria so that processing to recommend functional materials or bacteria suiting each user can efficiently be performed.

Moreover, an information processing apparatus to which the present invention is applied includes an acquisitor that acquires user information and a presenter that presents information about a functional material to the user, wherein the presenter presents the information about the functional material presented to the user acquired by the acquisitor based on information acquired after other users than the user take in the functional material.

Moreover, the information acquired after the other user takes in the functional material may be an evaluation with respect to the taken functional material done after the other user takes in the functional material.

Moreover, the information acquired after the other user takes in the functional material may be an examination result of a physical examination or excrement of the other user done after the other user takes in the functional material.

Moreover, the examination result of the excrement may be an examination result of intestinal bacteria of the other user.

Moreover, the presenter may present the information about the functional material presented to the user acquired by the acquisitor based on the evaluation of the functional material previously done by the user.

Moreover, an objective acquisitor (for example, the objective acquisitor 504 in FIG. 9) that acquires an objective of taking in the functional material by the user may be included and the presenter may change the information about the functional material sent to the user in accordance with the objective acquired by the objective acquisitor.

Moreover, a classifier (for example, the grouping unit 305 in FIG. 9) that classifies a plurality of users into two or more groups may be included and the other users may be selected from the same group as the user.

Moreover, the classifier may classify users using a degree of similarity of an intestinal bacterial flora.

Moreover, the functional material may include one or more lactic acid bacteria.

### REFERENCE SIGNS LIST

1 Server
2, 2-1, 2-n User terminal
101 CPU
102 ROM
103 RAM
104 Bus
105 Input/output interface
106 Outputter
107 Inputter
108 Storage
109 Communicator
110 Drive
120 Removable medium
201 User information acceptor
202 Recommendation result display
203 User feedback information generator
301 User information acquisitor
302 Effect index calculator
303 Recommender
304 User feedback information acquisitor
401 User DB
402 Material DB
403 Question DB
501 Comparator
502 Corrector
503 Recommendation target extractor
601 First corrector
602 Second corrector
603 Third corrector
U, U1, U2, U24, U25, Un User
N Network

1. An information processing apparatus comprising:
an acquisitor that acquires user information;
a presenter that presents information about a functional material including one or more lactic acid bacteria to the user; and
a classifier that classifies a plurality of users into groups based on information about an intestinal bacterial flora,
wherein the presenter presents the information about the functional material to the user acquired by the acquisitor based on information acquired after another user classified into a same group as the user by the classifier takes in the functional material and an evaluation of the functional material previously done by the user.

2. An information processing apparatus comprising:
an acquisitor that acquires user information; and
a presenter that presents information about a functional material to the user,
wherein the presenter presents the information about the functional material presented to the user acquired by the acquisitor based on information acquired after another user than the user takes in the functional material.

3. The information processing apparatus according to claim 2, wherein the information acquired after the other user takes in the functional material is an evaluation with respect to the taken functional material done after the other user takes in the functional material.

4. The information processing apparatus according to claim 2, wherein the information acquired after the other user takes in the functional material is an examination result of a physical examination or excrement of the other user done after the other user takes in the functional material.

5. The information processing apparatus according to claim 4, wherein the examination result of the excrement is an examination result of intestinal bacteria of the other user.

6. The information processing apparatus according to claim 2, wherein the presenter presents the information about the functional material presented to the user acquired by the acquisitor based on the evaluation of the functional material previously done by the user.

7. The information processing apparatus according to claim 2, further comprising:
an objective acquisitor that acquires an objective of the user to take in the functional material,
wherein the presenter changes the information about the functional material sent to the user in accordance with the objective acquired by the objective acquisitor.

8. The information processing apparatus according to claim 2, further comprising:
a classifier that classifies a plurality of users into two or more groups,
wherein the other user is selected from the same group as the user.

9. The information processing apparatus according to claim 8, wherein the classifier classifies users using a degree of similarity of an intestinal bacterial flora.

10. The information processing apparatus according to claim 1, wherein the functional material includes one or more lactic acid bacteria.

11. An information processing method comprising:
an acquisition step of acquiring user information; and
a presentation step of presenting information about a functional material to the user,
wherein in the presentation step, the information about the functional material presented to the user acquired in the acquisition step is presented to the user based on information acquired after another user than the user takes in the functional material.

12. A non-transitory computer readable storage medium storing a program for causing a computer to execute:
an acquisition step of acquiring user information; and
a presentation step of presenting information about a functional material to the user,
wherein in the presentation step, the information about the functional material presented to the user acquired in the acquisition step is presented to the user based on information acquired after another user than the user takes in the functional material.
